(19) United States
(12) Patent Application Publication
(10) Pub. No.: US 2026/0363681 A1
(43) Pub. Date: Nov. 19, 2026

(54) CERVICAL CELL COLLECTION DEVICE

(71) Applicant: Excell Company, LLC, Indianapolis, IN (US)

(72) Inventor: James A. Snyder, Carmel, IN (US)

(21) Appl. No.: 19/649,330

(22) Filed: Apr. 16, 2026

Related U.S. Application Data
(63) Continuation of application No. 16/999,595, filed on Aug. 21, 2020, now abandoned.

(51) Int. Cl. A61B 10/02

(52) U.S. Cl. CPC A61B 10/0291; A61B 2010/0216; A61B 2560/0406

(57) ABSTRACT

An exemplary device for collecting cervicovaginal cells includes a proximal end portion configured for gripping by a user, a distal end portion configured for contacting an ectocervical region and obtaining a sampling of cells therefrom, an elongated body portion extending between and connecting the proximal end portion and the distal end portion, and an elongated slot sized and shaped to receive a bristled portion of a brush having disposed thereon endocervical cells.

### TECHNICAL FIELD

The present disclosure generally relates to systems and methods for cell collection, and more particularly, but not exclusively, relates to systems and methods for collecting cervical cells for a liquid-based test.

### BACKGROUND

Detection and diagnosis of a variety of diseases, such as cancer, often involves the collection and microscopic examination of a cell sample. Such cell samples, as in the Papanicolaou (“Pap”) test, are typically collected from a patient with a type of cytology sampling device. Such sampling devices include various types of brushes and spatulas, as well as cotton swabs and loop-type devices.

In the case of brush type sampling devices, the cell samples were historically transferred after collection from the bristles of the brush to a glass slide that allows for the examination of the cells. In order to obtain the best diagnosis, it is necessary for the cell samples be completely and accurately transferred from the brush to the testing medium. Failure to achieve complete sample transfer can not only affect the diagnosis, but the opportunity to properly screen and provide care may be missed as women may be reluctant to have the procedure repeated when an inadequate sample is taken. It will be understood that an inadequate sample may include the instance where present abnormal cells are not effectively transferred to the glass slide, which results in false negatives.

In particular, it will be appreciated that the liquid-based Pap test methodology was introduced to address the shortcomings of sample transfer involving glass slides and is now the medical an industry standard used by doctors and labs. This involves the cell sample being transferred from the brush into a fixative or preservative within a vial or other container. One example of an FDA-approved liquid based preparation product is the ThinPrep® Pap Test™ (“TPPT”) made by Hologic Corporation of Marlborough, Massachusetts. As seen in the protocol for the TPPT, which is described in a ThinPrep® Pap Test Quick Reference Guide (1997), the brush is rotated vigorously while being pushed against the vial wall in order to release the cell sample in the solution. It will be appreciated, however, that pushing the cytology brush against the vial wall can have the detrimental effect of compacting the cell sample to the bristles. Additionally, the brush may not even touch the vial wall if the shaft of the brush is held in a certain way.

While the protocol set forth in the TPPT has been successful in gaining approval from the Food and Drug Administration, it has been found to be very operator dependent and results could be improved if a greater release of the cell sample from the brush is accomplished. One attempt at enhancing the transfer of a cell sample from a brush is disclosed in U.S. Pat. No. 6,394,966 to Gill et al., the contents of which are incorporated by reference in their entirety. While the device disclosed in the '966 patent has been shown effective in aiding in the transfer of cells from the brush to the liquid medium, certain difficulties were realized in the testing of the device. More particularly, it was found that the circular hole described therein provided a pinch point that could trap a portion of the brush and provided for uneven pressure against the bristles of the brush. For these reasons among others, there remains a need for further improvements in this technological field.

### SUMMARY

An exemplary device for collecting cervicovaginal cells includes a proximal end portion configured for gripping by a user, a distal end portion configured for contacting an ectocervical region and obtaining a sampling of cells therefrom, an elongated body portion extending between and connecting the proximal end portion and the distal end portion, and an elongated slot sized and shaped to receive a bristled portion of a brush having disposed thereon endocervical cells. Further embodiments, forms, features, and aspects of the present application shall become apparent from the description and figures provided herewith.

### BRIEF DESCRIPTION OF THE FIGURES

FIG. 1 is a plan view of a cell collection device according to certain embodiments.

FIG. 2 is a side view of the cell collection device illustrated in FIG. 1.

FIG. 3 is a plan view of a brush that may be utilized in certain embodiments.

FIG. 4 illustrates a kit according to certain embodiments, the kit including the cell collection device, the brush, and a vial containing a liquid preservative medium.

FIG. 5 is a plan view of a portion of the cell collection device illustrated in FIG. 1 when viewed at an angle perpendicular to a longitudinal axis of the device.

FIG. 6 is a plan view of an elongated slot according to certain embodiments.

FIG. 7 is a view of the same portion of the cell collection device illustrated in FIG. 5 when viewed at an angle oblique to the longitudinal axis of the device.

FIG. 8 is a schematic block diagram of a process according to certain embodiments.

FIG. 9 is a schematic block diagram of a process according to certain embodiments.

### DETAILED DESCRIPTION OF ILLUSTRATIVE EMBODIMENTS

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should further be appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described.

Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Items listed in the form of “A, B, and/or C” can also mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in certain specific arrangements and/or orderings. However, it should be appreciated that such specific arrangements and/or orderings may not necessarily be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may be omitted or may be combined with other features.

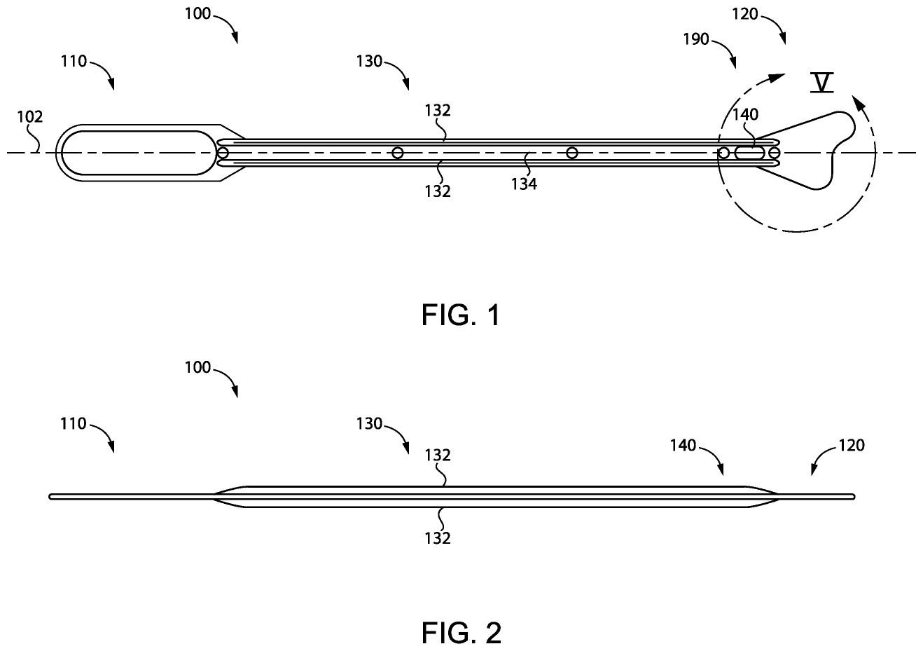

With reference to FIGS. 1 and 2, illustrated therein is a cell collection device 100 according to certain embodiments. The cell collection device 100 generally includes a proximal end portion 110 configured for gripping by a user, a distal end portion 120 configured for contacting an ectocervical region and obtaining a sampling of cells therefrom, an elongated body portion 130 extending between and connecting the proximal end portion 110 and the distal end portion 120, and an elongated opening or slot 140 sized and shaped to receive a bristled portion of a brush to aid in removing cells from the brush.

The proximal end portion 110 is configured for gripping by a user, and may further be configured for insertion into a vagina for collection of a vaginal wall cell sample. While other configurations are contemplated, in the illustrated form, the proximal end portion 110 is substantially planar.

The distal end portion 120 is positioned opposite the proximal end portion 110, and is configured for contacting the ectocervical region and obtaining a sampling of cells therefrom. While other configurations are contemplated, in the illustrated form, the distal end portion 120 is substantially planar.

The elongated body portion 130 extends between and connects the proximal end portion 110 and the distal end portion 120, and extends along a longitudinal axis 102 of the device 100. The illustrated body portion 130 generally includes a pair of rails 132 positioned on opposite sides of a planar wall 134 that extends along the longitudinal axis 102 to the elongated opening 140. As described herein, the rails 132 serve as a guide to aid insertion of a bristled portion of a brush into the elongated slot 140, as well as providing structural rigidity to the device 100.

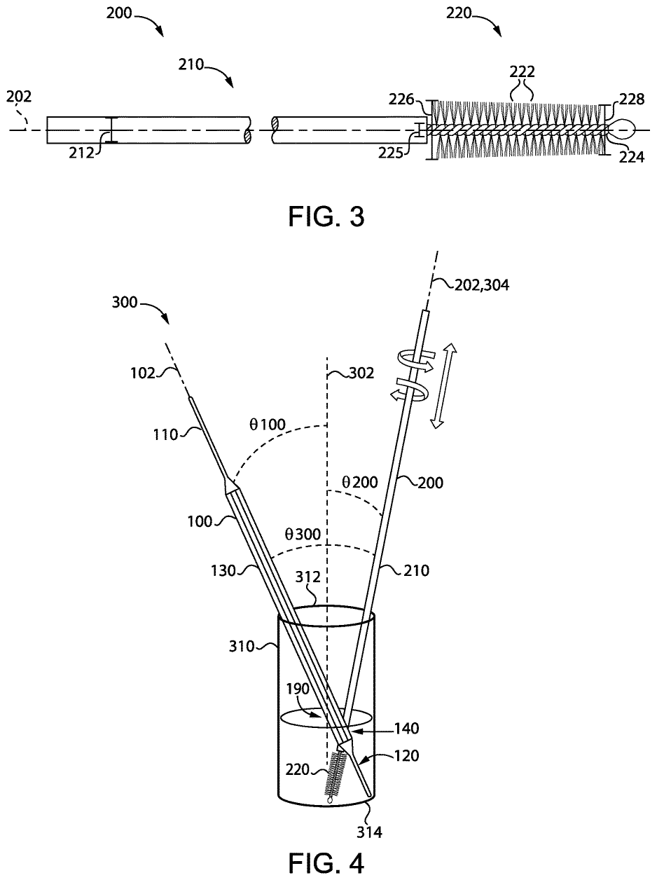

With additional reference to FIG. 3, illustrated therein is a cytology brush 200 with which the device 100 is configured for use. The brush 200 generally includes a handle or rod 210 and a bristled portion 220 positioned at a distal end of the rod 210.

The rod 210 extends along a longitudinal axis 202 of the brush 200, and provides a gripping region by which the brush 200 can be gripped by a user. The rod 210 has a width dimension or diameter 212.

The bristled portion 220 extends from the distal end of the rod 210, and includes a plurality of bristles 222 that project radially from a support shaft 224 having a support shaft diameter 224 less than the rod diameter 212. In the illustrated form, the bristled portion 220 is generally frustoconical in shape, and has a maximum diameter 226 and a minimum diameter 228 less than the maximum diameter 226. The maximum diameter 226 is greater than the rod diameter 212, and in the illustrated form, the minimum diameter 228 is also greater than the rod diameter 212.

With additional reference to FIG. 4, illustrated therein is a kit 300 according to certain embodiments. The kit 300 generally includes the device 100 and the brush 200, and may further include a vial 310 having a liquid preservative medium 320 disposed therein. During use of the kit 300, at least a portion of the device 100 is inserted into the vial 310 such that the distal end portion 120 and the elongated opening 140 are positioned within the liquid preservative medium 320. A portion of the device 100 is rested against an upper rim 312 of the vial 310, and the distal end portion 120 is rested against a lower rim 314 of the vial 310 such that the longitudinal axis 102 of the device 100 forms a first oblique angle θ100 relative to a vertical axis 302. The bristled portion 220 is inserted through the elongated opening 140, and the rod 210 may be rested on the upper rim 312 such that the longitudinal axis 202 of the brush 200 forms a second oblique angle θ200 relative to the vertical axis 302. The device 100 and the brush 200 are thus positioned such that the longitudinal axis 102 of the device 100 and the longitudinal axis 202 of the brush 202 define an oblique insertion angle θ300 relative to one another, with the brush 200 extending along an oblique insertion axis 304 relative to the device 100. The brush 200 may then be moved back and forth along the brush axis 202 while rotating the brush 200 about the brush axis 202 such that the bristles 222 engage and are teased by the edges of the elongated slot 140.

In the illustrated form, the first oblique angle θ100 is about 25°, and the second oblique angle θ200 is about 12° such that the insertion angle θ300 is about 37°. Those skilled in the art will readily recognize that these parameters can be achieved by appropriate selection of the vial height, the vial diameter, and the position of the slot 140 on the device 100. It should be appreciated that one or more of these parameters may be changed to alter one or more of the angles θ100, θ200, θ300. In certain embodiments, the oblique insertion angle θ300 may be between 25° and 45°. In certain embodiments, the oblique insertion angle θ300 may be between 35° and 40°. Other values for the oblique insertion angle θ300 are also contemplated as within the scope of the described subject matter.

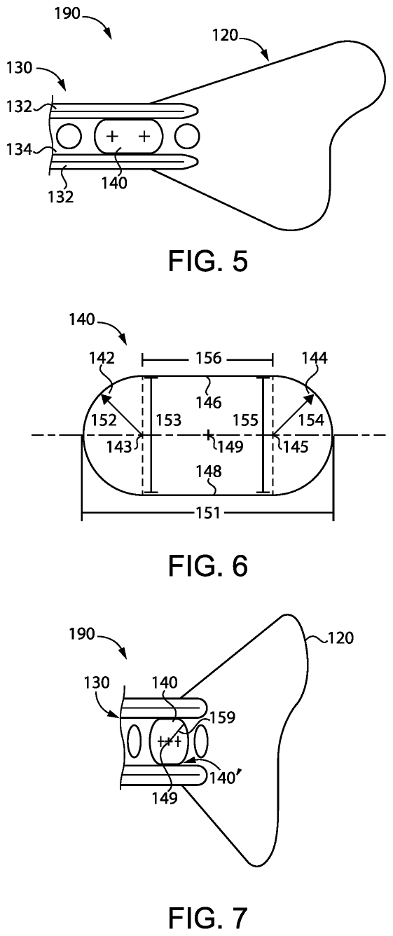

With additional reference to FIG. 5, illustrated therein is a plan view of the distal end portion 120, a distal portion of the elongated body 130, and the elongated slot 140. These portions of the device 100 are submerged in the liquid preservative medium 320 during use of the kit 300, and may be collectively referred to as the submergible portion 190. The view of FIG. 5 is one that is perpendicular to the longitudinal axis 102 and the plane defined by the distal end portion 102. In this view, the slot 140 is elongated along the longitudinal axis 102.

With additional reference to FIG. 6, the elongated slot 140 is defined by a plurality of edges, including a proximal curved edge 142, a distal curved edge 144, a first straight edge 146, and a second straight edge 148. The proximal curved edge 142 connects to proximal ends of the straight edges 146, 148, and the distal curved edge 144 connects to distal ends of the straight edges 146, 148 such that the straight edges 146, 148 extend between and connect the curved edges 142, 144 in a racetrack shape. While other forms are contemplated, in the illustrated form, each of the curved edges 142, 144 is semicircular about a corresponding and respective center point 143, 145, and has a corresponding and respective radius 152, 154.

The elongated slot 140 has a major axis 141 that is generally coincident with the longitudinal axis 102 of the device 100 and extends through a center point 149 of the slot 140. The slot 140 has a major diameter 151 extending along the major axis 141. The major diameter 151 includes the radius 152 of the proximal curved edge 142, the longitudinal length 156 of the straight edges 146, 148, and the radius 154 of the distal curved edge 144. The slot 140 has a proximal minor diameter 153 corresponding to twice the radius 152 of the proximal curved edge 142 and/or the offset distance between the proximal ends of the straight edges 146, 148. The slot 140 also has a distal minor diameter 155 corresponding to twice the radius 154 of the distal curved edge 144 and/or the offset distance between the distal ends of the straight edges 146, 148.

In certain embodiments, the longitudinal length 156 of the straight edges 146, 148 may be greater than one or both of the minor diameters 153, 155. In certain embodiments, the longitudinal length 156 of the straight edges 146, 148 may be equal to one or both of the minor diameters 153, 155. In certain embodiments, the longitudinal length 156 of the straight edges 146, 148 may be less than one or both of the minor diameters 153, 155.

In the illustrated form, the radius 152 of the proximal curved edge 142 is equal to the radius 154 of the distal curved edge 144 such that the straight edges 146, 148 are parallel to one another. It is also contemplated that the radius 152 of the proximal curved edge 142 may be greater than the radius 154 of the distal curved edge 144 such that the straight edges 146, 148 converge in a proximal-to-distal direction. In other embodiments, the radius 152 of the radius 152 of the proximal curved edge 142 may be less than the radius 154 of the distal curved edge 144 such that the straight edges 146, 148 diverge in a proximal-to-distal direction.

With additional reference to FIG. 7, illustrated therein is an oblique view of the submergible portion 190. More particularly, the oblique view illustrated in FIG. 7 is taken at the oblique insertion angle θ300 relative to the longitudinal axis 102 and the plane defined by the distal end portion 120. As will be appreciated, this view is that which would be seen by one looking along the brush axis 202 during insertion of the brush 200 into the slot 140 as described above. From this angle, the slot 140 no longer appears elongated, and instead appears to have a substantially circular shape 140′.

The slot 140, when viewed from the oblique insertion angle θ300, has a nominal radius 159, and the edges of the slot 140 are positioned within a predetermined distance of the center point 149 relative to the nominal radius 159. In other words, the edges are within an acceptable range of the nominal radius 159 relative to the center point 149. In certain embodiments, the acceptable range may be relatively low (e.g., 10% or 20%) such that the substantially circular shape 140′ is highly circular. In certain embodiments, the acceptable range may be higher such that the substantially circular shape 140′ is less circular.

The nominal radius 159 may be selected based at least in part upon the dimensions of the brush 200. It has been found that compatibility of a brush 200 with the device 100 is determined at least in part by the ability of the bristled section 220 of the brush 200 to pass safely through the slot 140 while the bristles 222 are safely and effectively teased, which allows for the transfer of sample cells trapped in the bristles 222. In embodiments in which the bristles 222 graduate in length, both the shorter and longer bristles should be considered.

In order to ensure compatibility with a particular embodiment of the brush 200, the dimensions of the slot 140 may be selected according to the dimensions of the brush 200. As one criterion for selecting dimensions of the slot 140, the minimum effective diameter of the slot 140, when viewed from the oblique insertion angle θ300, may be greater than the rod diameter 212 such that the distal end of the rod 210 can pass freely through the slot 140 at the oblique insertion angle θ300. Those skilled in the art will readily appreciate that it is desirable for the bristles 222 to be safely and effectively teased by the edges of the slot 140. As such, the dimensions of the slot 140 may be selected to ensure interference or overlap between the bristles 222 and the edges of the substantially circular shape 140′.

One commercially available embodiment of a brush 200 was analyzed to determine appropriate dimensions for the slot 140 in embodiments in which the device 100 is configured for use with such a brush. The brush 200 was found to have a maximum bristle diameter 226 of about 0.2704 inches, a minimum bristle diameter 224 of 0.2027 inches, a rod diameter 212 of 0.1128 inches, and a shaft diameter 225 of 0.0514 inches. Selecting the nominal diameter of the substantially circular shape 140′ as 0.1236 inches resulted in a maximum bristle overlap of 0.1468 inches (54%), a minimum bristle overlap of 0.0791 inches (39%), a shaft clearance of 0.0722 inches, and a rod clearance of 0.0108 inches. While these values demonstrate the efficacy of the selected dimensions for use with a particular embodiment of the brush 200, these values are provided as examples only. It should therefore be appreciated that other dimensions may be selected for the slot 140, both in embodiments configured for use with other forms of the brush 200 and embodiments configured for use with the same form of the brush 200.

Another parameter that may need to be selected is the location of the slot 140, both relative to the device 100 itself and relative to the level of liquid 320 in the vial 300. In the illustrated form, the slot 140 is provided to the elongated body portion 130, and more particularly between the rails 132. This arrangement has been found to have a positive effect on the structural integrity of the device 100. Relative to the level of liquid 320 in the vial 310, the slot 140 may be placed such that the slot 140 is just below the top fluid level to allow space for the bristled portion 220 to extend fully through the slot 140 without being obstructed by the base of the vial 310.

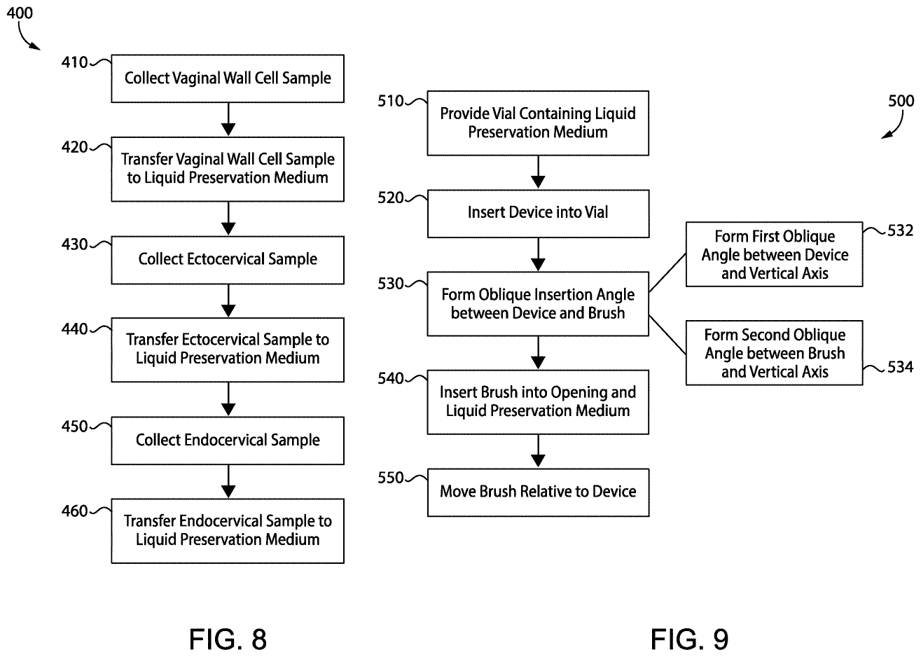

With additional reference to FIG. 8, an exemplary process 400 that may be performed using the kit 300 is illustrated. Blocks illustrated for the processes in the present application are understood to be examples only, and blocks may be combined or divided, and added or removed, as well as re-ordered in whole or in part, unless explicitly stated to the contrary. Additionally, while the blocks are illustrated in a relatively serial fashion, it is to be understood that two or more of the blocks may be performed concurrently or in parallel with one another. Moreover, while the process 400 is described herein with specific reference to the kit 300 illustrated in FIG. 4, it is to be appreciated that the process 400 may be performed with kits having additional or alternative components and/or features. It is also contemplated that the process 400 may be performed without a kit, for example in embodiments in which the device 100, the brush 200, and/or the vial 310 are provided separately and/or by different manufacturers.

The process 400 may begin with block 410, which generally involves collecting a vaginal wall cell sample. Block 410 may, for example, involve inserting the proximal end portion 110 of the device 100 into the vagina and performing a light scraping action on the vaginal wall. The process 400 may then continue to block 420, which involves transferring the vaginal wall sample to the liquid preservative medium 320. Block 420 may, for example, involve inserting the proximal end portion 110 into the liquid preservative medium 320 and stirring the medium 320 such that the sample transfers to the medium 320.

The process 400 may include block 430, which generally involves collecting an ectocervical sample. The ectocervical sample is collected from the ectocervix, resulting in the collection of primarily squamous cells. Block 430 may, for example, involve contacting the distal end portion 120 of the device 100 with the ectocervical area and rotating the device 100 to collect the cell sample from the ectocervix. The process 400 may then continue to block 440, which involves transferring the cell sample from the ectocervix to the liquid preservative medium 320. Block 440 may, for example, involve inserting the distal end portion 120 into the liquid preservative medium 320 and stirring the medium 320 such that the sample transfers to the medium 320.

The process 400 may further include block 450, which generally involves collecting an endocervical sample. During collection of the endocervical sample, the endocervical brush makes contact with the ectocervix, transformation zone, and endocervix, resulting in the collection of both squamous and glandular cells. Block 450 may, for example, involve inserting the bristled portion 220 of the brush 200 into the endocervical region and moving the brush 200 to collect the sample. The process 400 may then continue to block 460, which generally involves transferring the endocervical cell sample to the liquid preservative medium 320. Block 460 may, for example, proceed along the lines set forth below with reference to the process 500 illustrated in FIG. 9. Once one or more of the samples has been collected, the vial 310 may be sealed and sent to a lab for testing of one or more diseases or conditions.

With additional reference to FIG. 9, illustrated therein is a process 500 that may be performed to transfer a cell sample from a brush to a liquid preservative medium. The process 500 may, for example, be performed during block 460 of the above-described process 400 to transfer an endocervical sample from a brush 200 to a liquid preservative medium 320. While the process 500 is described herein with specific reference to the kit 300 illustrated in FIG. 4, it is to be appreciated that the process 500 may be performed with kits having additional or alternative components and/or features. It is also contemplated that the process 500 may be performed without a kit, for example in embodiments in which the device 100, the brush 200, and/or the vial 310 are provided separately and/or by different manufacturers.

The process 500 may begin with block 510, which generally involves providing a vial containing a liquid preservative medium. For example, block 510 may involve providing the above-described vial 310, which contains a liquid preservative medium 320.

The process 500 includes block 520, which generally involves inserting at least a portion of a device into the vial such that an elongated opening of the device is positioned within the liquid preservative medium. For example, block 520 may involve inserting the submergible portion 190 of the device 100 into the vial 310 such that the slot 140 is positioned within the liquid preservative medium 320.

The process 500 may include block 530, which generally involves forming an oblique insertion angle θ300 between a longitudinal axis 102 of the device 100 and a longitudinal axis 202 of a brush 200 such that the brush 200 defines an oblique insertion axis 304 relative to the device 100, wherein the brush 200 includes a bristled portion 220 that contains a cell sample.

In the illustrated form, block 530 includes block 532, which generally involves orienting the longitudinal axis 102 of the device 100 at a first oblique angle θ100 relative to a vertical axis 302. In certain embodiments, block 530 may involve positioning the device 100 such that a portion of the device 100 contacts an upper rim 312 of the vial 312 and the distal end portion 120 of the device 100 contacts the lower rim 314 of the vial 310, thereby orienting the longitudinal axis 102 of the device 100 at the first oblique angle θ100 relative to the vertical axis 302.

The illustrated form of block 530 further includes block 534, which generally involves orienting the longitudinal axis 202 of the brush 200 at a second oblique angle θ200 relative to the vertical axis 302. For example, block 534 may involve contacting the rod 210 with the upper rim 312 and moving the bristled portion 220 into engagement with the slot 140 such that the brush 200 defines the second oblique angle θ200 relative to the vertical axis 302.

In the illustrated form, block 530 involves orienting each of the device 100 and the brush 200 at a corresponding and respective oblique angle θ100, θ200 relative to the vertical axis 302 such that the oblique insertion angle θ300 is defined between the device 100 and the brush 200. It is also contemplated that one of the device 100 or the brush 200 may be held substantially vertical while the other of the device or the brush 200 is oriented at the oblique insertion angle θ300 relative to the vertical axis 302.

The process 500 further includes block 540, which generally involves inserting the brush 200 into the liquid preservative medium 320 and the elongated opening 140 along the oblique insertion axis 304, which is coincident with the brush axis 202. As noted above, the elongated slot 140 defines a substantially circular shape 140′ when viewed along the oblique insertion axis 304. As a result, engagement between the bristles 222 and the edges of the slot 140 is made more uniform and/or otherwise improved, and pinching of the brush 200 is reduced or eliminated.

As noted above, the blocks illustrated in the present application may be combined or divided, as well as reordered, and blocks may be performed in parallel with one another. For example, the orienting of block 530 may be performed in parallel with the inserting of block 540. By way of illustration, the process 500 may involve contacting the distal end of the brush 200 with the planar wall 134 of the body portion 130, and sliding the tip of the brush 200 along the wall 134 with the guidance of the rails 132 until the bristled portion 220 enters the slot 140, thereby concurrently performing the orienting of block 530 and the inserting of block 540.

The process 500 further includes block 550, which generally involves moving the brush 200 relative to the device 100 such that the bristled portion 220 engages one or more edges of the elongated slot 140, thereby releasing cells of the cell sample into the liquid preservative medium 320. Block 550 may, for example, involve reciprocating the brush 200 along the oblique insertion axis 304 and/or rotating the brush about the oblique insertion axis 304. Block 550 may further include removing the brush 200 from the opening 140 and swirling the submergible portion 190 in the medium 320 to provide for further transfer of cell matter from the edges of the opening 140 to the medium 320.

In testing, it was found that many users performing block 550 performed too few reciprocations, resulting in incomplete transfer of the cell sample from the brush 200, while other users performed too many reciprocations, which increased the risk of spillage. It has been found that the risk of incomplete transfer and/or spillage can be mitigated by expressly instructing the user to specifically perform a predetermined number of in and out repetitions (e.g., ten reciprocations) while also rotating the brush rod 210 between the fingers. As such, in certain embodiments, block 550 may involve reciprocating the brush 200 along the oblique insertion axis 304 a predetermined number of times. Instructing the user to perform a predetermined number of reciprocations may also serve to reduce inter-operator variability and ensure that all users have the opportunity to perform the transfer step with high effectiveness.

With the sample transferred to the preservative medium 320, the process 500 may be completed by removing the device 100 and brush 200 from the vial 310 and sealing the vial 310. The vial 310 may then be sent to a lab for testing. In certain embodiments, the sample may be subjected to Pap testing. It has been found that the improved transfer capabilities of the systems and methods described herein also provide for greater residual samples for ancillary molecular tests such as human papilloma virus (HPV), gonorrhea, chlamydia, herpes simplex virus (HSV), and others. The improved transfer capabilities improves overall specimen adequacy and may lead to an increase in detection of LSIL (Low-grade Squamous Intraepithelial Lesions), HSIL (High-grade Squamous Intraepithelial Lesions), and other more severe squamous abnormalities, as well as detection of abnormalities in glandular cells.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected.

It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

What is claimed is:

1. A device for collecting cervicovaginal cells, comprising:
a proximal end portion configured for gripping by a user;
a distal end portion configured for contacting an ectocervical region and obtaining a sampling of cells therefrom;
an elongated body portion extending between and connecting the proximal end portion and the distal end portion; and
an elongated slot sized and shaped to receive a bristled portion of a brush having disposed thereon an endocervical sample.

2. The device of claim 1, wherein the elongated slot has a racetrack shape.

3. The device of claim 1, wherein the elongated slot comprises a pair of straight edges having first ends and second ends, a first semicircular edge connected with the first ends of the straight edges, and a second semicircular edge connected with the second ends of the straight edges.

4. The device of claim 1, wherein the elongated body portion comprises a pair of parallel rails; and
wherein the elongated slot is positioned between the rails.

5. The device of claim 1, wherein the elongated slot is elongated along a longitudinal axis of the elongated body portion.

6. A kit comprising the device of claim 1 and further comprising the brush;
wherein the bristled portion of the brush has a maximum bristle diameter; and
wherein an edge of the elongated slot has a radius of curvature less than one half the bristle diameter.

7. A kit comprising the device of claim 1 and further comprising the brush;
wherein the bristled portion of the brush has a maximum bristle diameter; and
wherein the elongated slot is defined in part by a pair of parallel straight edges that are offset from one another by an offset distance less than the maximum bristle diameter.

8. A device for collecting cervicovaginal cells, comprising:
a proximal end portion configured for gripping by a user;
a distal end portion opposite the proximal end portion;
an elongated body portion extending along a longitudinal axis and connecting the proximal end portion and the distal end portion; and
an elongated opening having a racetrack shape, wherein the elongated opening has a circular shape when viewed at an oblique insertion angle relative to the longitudinal axis, wherein the oblique insertion angle is between 25° and 45°.

9. The device of claim 8, wherein edges of the circular shape are within 20% of a nominal radius of the circular shape relative to a center point of the circular shape.

10. The device of claim 8, wherein the racetrack shape comprises a pair of curved edges and a pair of straight edges extending between and connecting the curved edges.

11. The device of claim 10, wherein a length of the straight edges is less than a diameter of the curved edges.

12. The device of claim 8, wherein the elongated opening is elongated along the longitudinal axis.

13. A method for harvesting cells, the method comprising:
providing a vial containing a liquid preservative medium;
inserting at least a portion of a device into the vial such that an elongated slot of the device is positioned within the liquid preservative medium;
forming an oblique insertion angle between a longitudinal axis of the device and a longitudinal axis of a brush such that the brush defines an oblique insertion axis relative to the device, wherein the brush includes a bristled portion containing a cell sample;
inserting the bristled portion into the liquid preservative medium and the elongated opening along the oblique insertion axis, wherein the elongated slot is circular when viewed along the oblique insertion axis; and
moving the brush relative to the device such that the bristled portion engages one or more edges of the elongated slot, thereby releasing cells of the cell sample into the liquid preservative medium.

14. The method of claim 13, wherein the oblique insertion axis and the longitudinal axis of the device define an insertion angle between 25° and 45°.

15. The method of claim 13, wherein the elongated slot comprises a pair of curved edges and a pair of straight edges extending between and connecting the curved edges.

16. The method of claim 15, wherein the pair of curved edges are offset from one another along the longitudinal axis; and
wherein the pair of straight edges are parallel to the longitudinal axis.

17. The method of claim 16, wherein the curved edges are semi-circular in shape.

18. The method of claim 13, wherein forming an oblique insertion angle between the device and the brush comprises:
orienting a longitudinal axis of the device at a first oblique angle relative to a vertical axis; and
orienting the brush at a second oblique angle relative to the vertical axis such that the brush extends along the oblique insertion axis relative to the device.

19. The method of claim 18, wherein orienting the longitudinal axis of the device comprises positioning the device such that a portion of the device contacts an upper rim of the vial and a distal end portion of the device contacts a lower rim of the vial, thereby orienting the longitudinal axis of the device at the first oblique angle relative to the vertical axis.

20. The method of claim 13, wherein moving the brush relative to the device comprises reciprocating the brush along the oblique insertion axis a predetermined number of times.
